(19) United States
(12) Patent Application Publication
(10) Pub. No.: US 2026/0327350 A1
(43) Pub. Date: Oct. 22, 2026

(54) A DERIVATIVE OF TAMARIND SEED POLYSACCHARIDE AND A PREPARATION PROCESS THEREOF

(71) Applicant: FARMIGEA S.P.A., Pisa (IT)

(72) Inventors: Marco Aldo Sanso', Pisa (IT); Fabio Neggiani, Pisa (IT)

(73) Assignee: FARMIGEA S.P.A., Pisa (IT)

(21) Appl. No.: 19/122,798

(22) PCT Filed: Oct. 23, 2023
(86) PCT No.: PCT/IB2023/060682

§ 371 Date: Apr. 21, 2025

(30) Foreign Application Priority Data
Oct. 26, 2022 (IT) 102022000022101

(51) Int. Cl. C08B 37/00; A61K 47/36

(52) U.S. Cl. CPC C08B 37/0087; A61K 47/36

(57) ABSTRACT

A derivative of tamarind seed polysaccharide (TSP) is disclosed, and a process for its preparation. In particular, said derivative is a sulphated TSP having a selected sulphation degree and molecular weight, showing improved workability, stability and compatibility with pharmaceutical ingredients. Therefore, a pharmaceutical composition, as well as a biomaterial, comprising said sulphated TSP are also disclosed. Additionally, the cosmetic use of the sulphated TSP is reported. Finally, a process of preparation of said sulphated TSP is described.

### FIELD OF THE INVENTION

The present invention relates to a derivative of tamarind seed polysaccharide (TSP), and a process for its preparation. In particular, said derivative is a sulphated TSP having a selected sulphation degree and molecular weight, showing improved workability, stability and compatibility with pharmaceutical ingredients.

Therefore, the invention also relates to a pharmaceutical composition, as well as a biomaterial, comprising said sulphated TSP.

Finally, the present invention also concerns a process of preparation of said sulphated TSP.

### BACKGROUND ART

The term “tamarind seed polysaccharide” means the polysaccharide moiety obtainable from the seeds of Tamarindus indica, also referred to hereinafter for the sake of brevity as “TSP” (from the English term “Tamarindus indica Seed Polysaccharide”).

As it is known, the tamarind tree is common in India, in Africa, and throughout the Far East, where it is grown essentially for food purposes. The seed, which was originally a by-product, has since found various uses, sometimes ground up into a mealy product (currently known as “raw tamarind gum or “tamarind nut powder”), above all in the textile and paper industry, where it is used respectively as a sizing agent for yarn and as a gluing agent, and in the food industry, where it is used as a thickener, gelling agent, stabiliser and binder in all kinds of products, much the same ways as further polysaccharide products, such as alginates, pectins, the guar gum or the locust bean meal. Raw tamarind gum (commercially available, for example, as Glyloid® produced by Dai-nippon Pharmaceutical Co. LTD based in Osaka, Japan) typically contains, in addition to 65-73 wt % polysaccharide, also 15-23% protein material, 3-8% oils and fats and 2-4% ash, as well as traces of raw fibre, tannins, and further impurities.

One advantageous aspect is that the TSP solutions are suitable to be sterilized by a passage in autoclave (for example for 20 minutes at 120° C.) without undergoing thermal degradation, unlike as occurs, for example, with hyaluronic acid. The possibility of sterilization by simply a passage in autoclave renders the TSP-based preparations particularly convenient from a production viewpoint.

Furthermore, TSP has demonstrated significant mucomimetic, mucoadhesive, and bioadhesive properties.

TSP (or “native TSP”) is a purified, neutral, water-soluble polysaccharide fraction comprising a polymeric molecule of galactoxyloglucan which is very hydrophilic and features a ramified structure: attached to the main linear chain, formed of glucose repeating units, are small monosaccharide units of xylose and disaccharide units of xylose-galactose, in the latter case, the galactose is at the end of the side chain. The three monomers are present in a molar ratio of 3:1:2 and constitute approximately 65% of the components of the seed, as illustratively shown in FIG. 1. As can be observed, the “mucin-like” molecular structure determines the excellent mucoadhesive properties of the polysaccharide, derived from the formation of bonds, of various kinds, with said mucins. TSP can be isolated by means of chemical methods and enzymatic methods, by using protease or a combination of protease and high intensity ultrasounds. In the chemical method, Tamarind seed powder are added to cold distilled water to prepare a suspension which is then poured into boiling distilled water. The solution thus formed is left to boil and stirred continually; after resting for one night, said solution, undergoes centrifugation. The supernatant is separated and poured into a volume of pure alcohol amounting to double the amount of said supernatant. Thus, a precipitate is obtained which is then washed with pure ethanol and air-dried. Finally, the dried polymer is ground up, sieved, and stored in a dryer until use. In the enzymatic method, the powder obtained from the seeds is mixed with ethanol and then treated with protease; subsequently, said powder is centrifuged and ethanol is added to the supernatant for precipitation. Finally, the polymer is separated and dried.

Natural polymers, such as TSP, have advantages over synthetic and semi-synthetic polymers like low cost, natural origin, less side effects, locally availability and higher patient tolerance.

However, these natural substances also suffer with the drawbacks like purity, source and microbial contamination. At the same time, TSP has the additional problem of poor workability, due to its complex structure and requires long pre-treatment in order to be suitable for subsequent preparations, as above reminded. This is confirmed also by a very recent publication of Effendi A. D. et al. dated March 2022 (“Polysaccharides from Tamarindus indica L. as natural kinetic hydrates inhibitor at high subcooling environment”, Journal of Petroleum Exploration and Production Technology (2022) 12:2711-2722), wherein, in p. 2714, a very complex and time-consuming procedure is disclosed for extracting TSP from Tamarind seed powder. In particular, the authors report the following procedure:

“Initially, 20 g of TSP powder was soaked for 24 h in 800 mL of distilled water. To ensure the release of mucilage into the distilled water, the mixture was cooked for 1 h at 100° C. and then set aside for 2 h. To remove all of the foreign materials, the solution was centrifuged at 6000 rpm for 20 min. The supernatant was separated from the rest of the mixture. For mucilage precipitation, an equal amount of acetone was added to the supernatant. The precipitate was collected using a stainless filter and dried for 4 h at 50° C. in an oven. The dried polymer was kept in a desiccator until it was needed again. The dry polymer was mixed with distilled water to make polysaccharides inhibitors at various concentrations.” The authors explain that tamarind xyloglucan has a similar property to other polysaccharides in that it is still water soluble even when its individual chains are not fully hydrated, but it has been able to form a colloidal dispersion in aqueous solution due to a balanced hydrophobic and hydrophilic properties, so that this is likely the reason underlying a so long and complex procedure.

Later on, in p. 2715, the authors add that “Polysaccharides are economical and easy to obtain, but they are typically insoluble in water or have a high viscosity after dissolving in water, making them difficult to inject into a pipeline during use. It must either increase its water solubility or develop a new injection procedure (Wang et al. 2019). The solubility is related to the hydrophilic and hydrophobic chain present in the polymer. A hydrophilic dominant polymer would allow better solubilisation which indirectly relate to its inhibition mechanisms through adsorption. Hence, the solubility of TSP is important for it allows the TSP to mix with the multiphase fluid composing mainly of water and hydrocarbon. Therefore, the dried polysaccharides were subjected to a solubility test using distilled water as the solvent in two separate conditions: room temperature water and boiling water.” To this end, in p. 2717, the following procedure has been described: “As polysaccharides usually have low solubility, the dried TSP were subjected to a solubility test using distilled water as the solvent in two separate conditions: room temperature water and boiling water. The dried polymer appears to be sparingly soluble in room temperature water but quickly solubilise in boiling water according to the investigation. The result indicates that TSP require solubilisation with boiling water to effectively inject into hydrate prone location. Solubilisation of TSP is closely related to KHI performance since efficient KHIs depends on the adsorption affinity of KHIs. Therefore, TSP efficiency can further improve by grafted by hydrophilic group improvement or the use of microcapsule.” The authors concluded in p. 2720 that: “TSP appears to be sparingly soluble in room temperature water but quickly solubilise in boiling water. This information helps operator to solubilise the polymer into higher temperature fluid when injecting, as the purpose is to prevent hydrate formation, the additional high temperature of fluid injected would contribute to the hydrate prevention technology to encapsulate.”

However, it should be considered that Effendi et al. refer to a specific technical field, as clear e.g. from the Abstract (“In an offshore system, hydrocarbon fluids are produced at deeper depths in the oceans, and extended pipelines delivering fluids over long distances are common. Subsequently, these practices increase the tendency of unprocessed water-containing hydrocarbon fluid to be exposed to lower temperatures and higher pressures conditions where hydrate formation is favourable. One of the solutions to resolve this problem is by introducing hydrate inhibitors preferably low dosage hydrate inhibitors (LDHIs). The more versatile LDHIs; Kinetic Hydrate Inhibitors (KHIs) could further be optimised in cost and its biodegradation properties. [ . . . ] The outcomes shows that TSP is able to delay hydrates formation at high degree of subcooling. The TSP works well at low concentration at high degree of subcooling while remain relatively economical and biodegradable.”)

Actually, the solution proposed and described in Effendi et al., i.e. boiling TSP in water and/or using microcapsule technology, cannot be borrowed in the pharmaceutical field, where the requirements are more stringent and TSP should be worked without negatively affecting its intrinsic characteristics. Indeed, high temperatures result in the depolymerization of the TSP mucopolysaccharide chains, especially when exposure to high temperatures lasts for prolonged periods of time. This degradation makes TSP not applicable for pharmaceutical formulations, as the degradation products would render the pharmaceutical preparations highly unstable and ineffective. Moreover, the encapsulation method could be proper only in case of TSP having low molecular weight, which is not the case of pharmaceutical applications.

In the inventors' experience, dried TSP, such as freeze-dried TSP, needs to be redissolved in water at room temperature in order to maintain its chemical-physical properties and then be usable for further preparations, resulting in dried TSP to be soaked in water for hours, i.e. minimum for 6 hours. Actually, the purer the dried TSP and the higher the molecular weigh of TSP, the longer the soaking time for restoring a properly rehydrated TSP.

Therefore, it is an object of the present invention to improve the overall workability and economical convenience of TSP within the context of pharmaceutical productions, while at the same time achieving a good stability overtime and higher compatibility with other pharmaceutical ingredients.

### SUMMARY OF THE INVENTION

Said object has been achieved by a sulphated tamarind seed polysaccharide (TSP), as stated in claim 1.

In a further aspect, the present invention concerns a pharmaceutical composition comprising said sulphated TSP.

In a further aspect, the present invention concerns a biomaterial comprising said sulphated TSP.

In another aspect, the present invention concerns the cosmetic use of said sulphated TSP.

In a further aspect, the present invention concerns a process of preparation of said sulphated TSP.

### BRIEF DESCRIPTION OF THE FIGURES

The characteristics and advantages of the present invention will become clear in the following detailed description of the embodiments provided by way of non-limiting examples and illustrated in the drawings annexed hereto, wherein:

FIG. 1 shows the structure of the octasaccharide repeating unit of the native tamarind seed polysaccharide (shortly “TSP”),

FIG. 2 shows ¹H NMR spectrum of native TSP,

FIG. 3 shows HSQC-DEPT spectrum of native TSP,

FIG. 4 shows HSQC-DEPT spectra of native TSP and a sulphated TSP according to the invention, as per Example 1,

FIG. 5 shows the comparison between the ¹³C-NMR spectra of the native TSP and the sulphated TSP according to the invention, as per Example 1,

FIG. 6 shows the viscosity values as a function of the shear rate of the sulphated TSP according to the invention, as per Example 1,

FIG. 7 shows HSQC-DEPT spectra of native TSP and a sulphated TSP according to the invention, as per Example 2,

FIG. 8 shows the comparison between the ¹³C-NMR spectra of native TSP and a sulphated TSP according to the invention, as per Example 2,

FIG. 9 shows the variations of viscosity as a function of the shear rate of a sulphated TSP according to the invention, as per Example 2,

FIG. 10 shows HSQC-DEPT spectra of native TSP and a sulphated TSP according to the invention, as per Example 3,

FIG. 11 shows the comparison between the ¹³C-NMR spectra of native TSP and a sulphated TSP according to the invention, as per Example 3, and

FIG. 12 shows the variations of viscosity as a function of the shear rate of a sulphated TSP according to the invention, as per Example 3.

### DETAILED DESCRIPTION OF THE INVENTION

The invention therefore relates to a sulphated tamarind seed polysaccharide (TSP) having a weight average molecular weight of 600-1′500 kDa, and a degree of sulphation (DS) of 1 to 10 per octasaccharide repeating unit, said DS being determined via conductimetric titration and calculated according to the following formula:

$$D {S}_{( m o l e )} = \frac{mmole NaOH}{\frac{mg TSP - ( mmole NaOH \times 80 g / mole )}{1207 g / mole}}$$

where NaOH is the titrating agent.

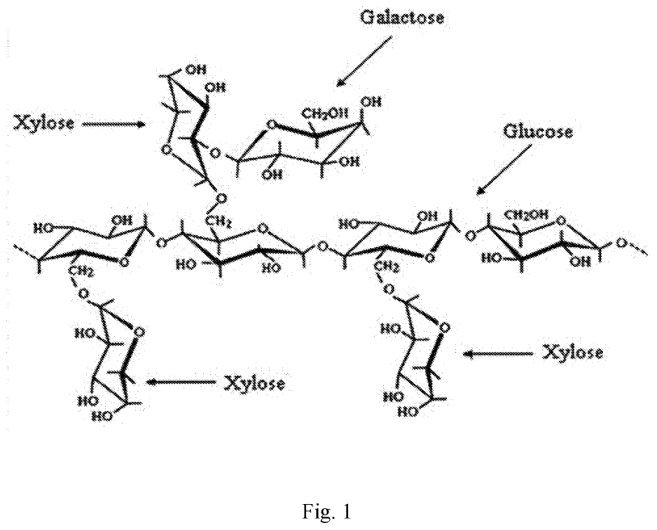

In particular, the degree of substitution (DS) indicates the average number of sulphonic groups linked to octasaccharide repeating unit of the native TSP, and is calculated by the above formula, where “1207 g/mole” is the weight of an average repeating unit of TSP and “80 g/mole” is the weight difference between the group —OS₃H and the group —OH. As said above and as shown in FIG. 1, TSP is constituted by saccharide units, including α-D-xylopyranose, β-D-galactopyranose and glucose, bound via glycosidic linkages to form a branched polymer.

The combination of selected DS range of 1 to 10 and weight average molecular weight has been observed to give an advantageous balance between sulphation degree and preservation of the starting TSP backbone. In fact, when sulphation degree higher then 10 have been imparted, an undesired depolymerization of TSP has been noticed, while sulphation degrees lower than 1 have no appreciable impact on the TSP properties.

Additionally, it has been surprisingly found that the dried sulphated TSP needs to be soaked in water for only 3 hours at room temperature, in order to be redissolved and usable for further preparations. This means that, once sulphated at the above indicated degree, TSP results to be more workable and stable overtime, as well as more compatible with other pharmaceutical ingredients.

Preferably, the sulphated tamarind seed polysaccharide has a degree of sulphation (DS) of 1 to 5 per octasaccharide repeating unit.

The molecular weight of sulphated TSP is another important parameter influencing its properties as well as bioactivities. The range indicated above means that the process of sulfation is successfully carried out without degradation or depolymerization of the starting TSP.

Preferably, the sulphated tamarind seed polysaccharide has a weight average molecular weight of 700-1′100 kDa.

Preferably, the sulphated TSP of the invention has a viscosity of 20 to 60 mPa*s, at a shear rate of 10 l/s and at a temperature of 20° C. It should be noted that the corresponding starting TSP has a viscosity of 80 mPa*s or higher, therefore appreciably the sulphated TSP shows a reduced viscosity, making the same more workable.

Preferably, the sulphated TSP of the invention has a Zeta potential of −20.00 to −50.00 mV, as measured by Dynamic Light Scattering (DLS). It should be noted that the corresponding starting TSP has Zeta potential of −0.36 mV or higher (i.e. a neutral polymer), therefore appreciably the sulphated TSP is a negatively charged polymer, making the same more compatible with water-soluble (or polar solvent-soluble) ingredients.

In some embodiments, the sulphated TSP is salified with a heavy metal or with a pharmacologically active substance.

Preferably, said heavy metal is a metal element selected from the 4th, 5th and 6th groups of the periodic table of elements.

More preferably, the heavy metal is silver, cobalt, iron, copper, zinc, arsenic, strontium, zirconium, antimony, gold, caesium, tungsten, selenium, platinum, ruthenium, bismuth, tin, titanium, or mercury.

Preferably, said pharmacologically active substance is selected from the group consisting of an antibiotic, an anti-infective, an antimicrobial, an antiviral, a cytostatic, an antitumoral, an anti-inflammatory, a wound healing agent, an anaesthetic, a cholinergic antagonist, an adrenergic agonist, an antithrombotic, an anticoagulant, a haemostatic, a fibrinolytic, a thrombolytic agent, a protein, a protein fragment, a peptide, and a polynucleotide.

In another aspect, the present invention concerns a pharmaceutical composition comprising the sulphated tamarind seed polysaccharide above described and pharmaceutically acceptable excipients.

The term “excipient” means a compound or a mixture of compounds suitable for pharmaceutical use, respectively. For example, an excipient for use in a pharmaceutical grade formulation generally must not cause an adverse response in a subject, nor must it significantly inhibit the efficacy of the sulphated TSP contained therein. Suitable excipients are acidifiers, acidity regulators, anti-caking agents, antioxidants, bulking agents, resistance agents, gelling agents, glazing agents, modified starches, sequestrants, thickeners, sweeteners, thinners, disaggregants, glidants, dyes, binders, lubricants, stabilisers, adsorbents, humectants, flavours, film-forming substances, emulsifiers, wetting agents, release retardants and mixtures thereof. Preferably, said excipients are olive oil, mineral oil, liquid paraffin, white petrolatum, polyoxyethylene, emulsifying wax, stearyl alcohol, isostearyl alcohol, cetylstearyl alcohol, stearic acid, glyceryl stearate, sodium lauryl sarcosinate, glycerine, diethylene glycolmonoethyl ether, polyethylene glycol, polyethylene glycol, polyethylene glycol stearates, Carbopol, carbomers, Poloxamer 407, Macrogol 400, purified bentonite, myristyl propionate, dimethicone, titanium dioxide, anionic, cationic and non-ionic surfactants, water, potassium sorbate, sodium benzoate, F-polylysine, sucralose, maltodextrin, citric acid, sodium carbonate, calcium carbonate, magnesium carbonate, magnesium stearate, natural starch, partially hydrolysed starch, modified starch, lactose, calcium phosphate, calcium carbonate, calcium sulfate, polyvinylpyrrolidone, silica, colloidal silica, precipitated silica, magnesium silicates, aluminium silicates, sodium lauryl sulfate, magnesium lauryl sulfate, methacrylate copolymers, sodium dehydroacetate, xanthan gum, guar gum, tara gum, carob gum, fenugreek gum, Arabic gum, alginic acid, sodium alginate, propylene glycol alginate, sodium croscarmellose, polyvinylpolypyrrolidone, glyceryl behenate, indigo carmine, cellulose, modified cellulose, calcium carboxymethylcellulose, sodium carboxymethylcellulose, microcrystalline cellulose, ethyl cellulose, gelatine, hydroxyethyl cellulose, hydroxypropyl cellulose, polydextrose, carrageenan, methylcellulose, sucrose, sucrose esters, sorbitol, xylitol, dextrose, maltitol, tragacanth gum, pectin, agar-agar, carboxypolymethylene, hydroxypropyl methylcellulose, tragacanth gum, mannitol, or mixtures thereof.

In another aspect, the present invention relates to a biomaterial comprising the sulphated tamarind seed polysaccharide as above described, and a natural, a semisynthetic or a synthetic polymer, wherein the natural polymer is selected from the group consisting of a collagen, a coprecipitate of collagen and glycosaminoglycan, a cellulose, a polysaccharides in the form of a gel selected from the group consisting of chitin, chitosan, pectin, pectic acid, agar, agarose, xanthan, gellan, alginic acid, an alginate, polymannan, a polyglycan, starch, and a natural gum, wherein the semisynthetic polymer is a collagen crosslinked with a crosslinking agent selected from the group consisting of an aldehyde, a precursor of an aldehyde, a dicarboxylic acid, a dicarboxylic acid halogenide, a diamine, a cellulose derivative, hyaluronic acid, chitin, chitosan, gellan, xanthan, pectin, pectic acid, a polyglycan, polymannan, agar, agarose, natural gum and glycosaminoglycans, and wherein the synthetic polymer is selected from the group consisting of polylactic acid, polyglycolic acid, a copolymer of polylactic acid, a derivative of polylactic acid, a copolymer of polyglycolic acid, a derivative of polyglycolic acid, polydioxan, polyphosphazene, a polysulphonic resin, polyurethane and PTFE.

In a further aspect, the present invention concerns the cosmetic use of the sulphated tamarind seed polysaccharide as above described, as thickener, gelling agent, stabiliser, moisturizing agent, solubilizer, and/or smoothing agent.

When the sulphated TSP of the invention is used in a cosmetic product, the latter is preferably in the form of a solution, lotion, emulsion, suspension, gel, ointment, cream, paste, solution spray, transdermal patch, spray-on patch, foam, or wet wipe, wherein the composition is preferably a suspension or is dissolved in one or more suitable excipients. Examples of suitable cosmetically acceptable excipients are mineral oil, liquid paraffin, white vaseline, propylene glycol, polyoxyethylene, polyoxypropylene, emulsifying wax, stearyl alcohol, isostearyl alcohol, cetylstearyl alcohol, stearic acid, glyceryl stearate, sodium lauryl sarcosinate, glycerine, diethylene glycol monoethyl ether, polyethylene glycols, polyethylene glycol stearates, starch, carbopol, carbomers, methyl paraben, Poloxamer 407, Macrogol 400, purified bentonite, propyl paraben, myristyl propionate, dimethicone, titanium dioxide, anionic, cationic and non-ionic surfactants, water, and mixtures thereof.

The sulphated TSP of the invention can be prepared according to the process comprising the steps of:
i) providing a suspension of TSP in a tertiary amine solvent,
ii) adding a complex of sulphur trioxide with an organic radical selected from the group consisting of pyridine, DMF, trimethylamine, dioxane, N,N-dimethylaniline, B′,B-dichlorodiethyl ether, and mixture thereof, under stirring at a temperature of 15-60° C., thus obtaining a dispersion of sulphated TSP,
iii) adding water to said dispersion, thus obtaining a homogenous mixture, and adjusting the pH to 6-7,
iv) adding a alcoholic solvent to said mixture, for promoting the precipitation of the sulphated TSP, and
v) separating and purifying the sulphated TSP.

This preparation process allows to obtain the sulphated TSP of the invention at a yield of at least 90%.

In step i), TSP preferably in the form of powder, is suspended in a tertiary amine solvent, which enhances the nucleophilicity of the hydroxy groups on TSP backbone.

Preferably, said tertiary amine solvent is selected from DMF (dimethylformamide), pyridine, trimethylamine, picolines, N,N-dimethylaniline, quinolines, and mixtures thereof.

In preferred embodiments, said tertiary amine solvent is DMF, pyridine, trimethylamine, or a mixture thereof.

Preferably, the weight average molecular weight of the (native) TSP of step i) is 450-750 kDa, more preferably, 550-700 kDa. Actually, TSP having lower molecular weights tend to depolymerize, so that a sulphation reaction could further promote degradation, while TSP having higher molecular weights are in principle less suitable for pharmaceutical applications.

In step ii), a complex of sulphur trioxide is added to the suspension of TSP of step i).

In particular, said sulphur trioxide is complexed with an organic radical selected from the group consisting of pyridine, DMF, trimethylamine, dioxane, N,N-dimethylaniline, B′,B-dichlorodiethyl ether, and mixture thereof.

The complex acts as sulphating agent, i.e. sulphate groups are transferred to TSP, while releasing the organic radical as the corresponding neutral compound, which can be used as a solvent in subsequently carrying out the process.

Step ii) is performed under stirring at a temperature of 15-60° C., thus obtaining a dispersion of sulphated TSP. Preferably, the temperature is room temperature, i.e. 20-25° C., however, when a higher temperature is selected, the resulting dispersion of sulphated TSP is left to cool down to room temperature, before performing step iii).

Preferably, in step ii) said complex and TSP are in a molar ratio of 1:1 to 1:10, more preferably 1:1 to 1:5. It has been experimentally observed that the higher the ratio between the sulphating agent and TPS, the greater the undesired TSP depolymerization, therefore these ranges give a better balance between sulphation degree and preservation of the starting TSP backbone.

In step iii), water is added to said dispersion, thus obtaining a homogenous mixture, and the pH adjusted to neutral, i.e. 6-7.

The pH can be adjusted by adding an inorganic base, such as NaOH, KOH or NH₄OH.

In step iv), an alcoholic solvent is added to the homogeneous mixture obtained in step iii), for promoting the precipitation of the sulphated TSP.

Preferably, said alcoholic solvent is methyl alcohol, ethyl alcohol, propyl alcohol, or a mixture thereof, optionally diluted with water.

In step v), the precipitated sulphated TSP is separated and purified.

Preferably, the separation is performed by filtration, centrifugation or a combination thereof.

Preferably, the separated sulphated TSP is then purified, by dissolution in water, neutralisation to pH 6-7, and re-precipitation through addition of an alcoholic solvent, which can be the same as of step iv). The purification allows to further remove the residues of solvents, neutral compounds deriving from the complex, unreacted reagents and inorganic salts.

The barium test was used to determine the complete removal of sodium sulfate. Briefly, an aqueous solution of barium chloride (10% w v) was added to the aqueous solution of sulphate product. If a precipitate formed (barium sulfate which is almost insoluble in water, 2.5×10⁻³ g/L), there was sodium sulfate in the solution. If the solution remains clear, it indicates that the inorganic salt (sodium sulphate) has been completely removed. In preferred embodiments, the sulphated TSP resulting from step v) is then freeze-dried, in order to be long-term stored.

In the most preferred embodiments, the preparation process is performed as follows. Tamarind seed polysaccharide (TSP-500 mg) is suspended in dry dimethylformamide (DMF, 50 ml), in a three-neck flask at a selected temperature, stirred overnight at room temperature, and then, sulphur trioxide-pyridine complex at different mole/residue ratios of SO₃-py/polysaccharide (1:1, 1:2 and 1:4) is added. The resulting dispersion is stirred vigorously for 24 h at different temperature (r.t or 50° C.). After dilution with water and cooling to room temperature, a homogeneous mixture was obtained, the pH adjusted to 6-7 with NaOH solution (1N), and then added with alcohol (EtOH/H₂O 70% v/v) to precipitate the sulphated TSP, which is recovered by centrifugation. The solid sulphated TSP is then dissolved in water and after careful neutralisation with sodium hydroxide solution, precipitated again with ethanol 70% v/v to remove DMF, Pyridine and salt excess of sulphating agent and potential degradation production of the latter (Na₂SO₄) before being freeze-dried.

The kinetic of TSP sulphation was studied at 50° C. by varying the reaction time of step ii): 15 min., 24 and 48 h. It was observed that a reaction time of 24-48 h is preferable, as the sulphated TSP so obtained is advantageously preserved from chemical depolymerization, which occurs as a consequence of prolonged exposure to acidic conditions.

Moreover, the effect of temperature reaction was studied, by testing step ii) at different temperatures: 25, 50, 60 and 95° C. It was observed that a temperature of 15-60° C. is preferable, as it was found to minimize the risk of depolymerization of TSP backbone. Also, without wishing to be bound by any theory, it is believed that this temperature range allows to provide a regular and homogenous distribution of the sulphate group along the TSP chains, especially in high molecular weight TSP. More preferably, the temperature of step ii) is about 50° C.

In preferred embodiments, step ii) is performed at a temperature of 15-60° C., for 24-48 h. Finally, the effect of sulphating agent was assessed by testing different complexes, i.e. sulphur trioxide pyridine complex (shortly “SO₃*Py”), sulphur trioxide trimethylamine complex (shortly “SO₃*Me₃N”) and sulphur trioxide N,N′dimethylformamide complex 5 (shortly “SO₃*DMF”), all showing satisfactory performances in sulphating TSP.

It should be also understood that all the combinations of preferred aspects of the sulphated TSP, as well as of the products containing the same, their preparation and uses, as above reported, are to be deemed as hereby disclosed, and similarly preferred.

It should also be also understood that all combinations of the preferred aspects of the sulphated TSP of the invention, preparation processes, and uses disclosed above are to be understood as herein described.

Below are working examples of the present invention provided for illustrative purposes.

### EXAMPLES

### Materials

Samples and Reagents used in the following examples are described hereafter in table 1 and table 2, respectively.

TABLE 1

| Code | Samples |
|---|---|
| P7300 | Tamarind Seed Polysaccharide (TSP) |
| P7351 | Sulphated derivative of TSP |
| P7352 | Sulphated derivative of TSP |
| P7353 | Sulphated derivative of TSP |

TABLE 2

| Compound | Supplier |
|---|---|
| Deionized water | Culligan system |
| Dimethylformamide (DMF) | Sigma Aldrich |
| dry over molecular sieve | |
| Dimethylformamide-sulphur trioxide (SO₃ × DMF) | Sigma Aldrich |
| Sulphur trioxide pyridine complex (SO₃ × Py) | Sigma Aldrich |
| Sodium Hydroxide 0.1N | Sigma Aldrich |

### Equipment

The instruments used in the following examples are reported hereafter:
Heating plate: IKA RCT basic and IKA C-MAG HS 7;
Thermo Shaker: PSC32, PHMT Grant-bio;
Vortex: Velp Scientifica.
Zetasizer Nano ZS (Malvern Panalytical);
Rheometer: Modular Compact Rheometer MCR 92Anton Paar.
HP-SEC-TDA system: model Viscotek-TDA 302
NMR: Bruker Avance 500 Neo instrument, equipped with 5 mm cryoprobe
888 Titrando system

### Methods

Zeta Potential (Zp) of molecules in the tested samples has been evaluated by Dynamic Light Scattering (DLS) Technique, by using the following parameters:

### Zeta Potential

Material: Polystyrene Latex; Dispersant: Water; cuvettes: disposable polystyrene cuvettes; Temperature: 40° C.; measures: 3; runs: 10; delay between measures: 10 s; type of acquisition: General Purpose. Temperature: 40° C.

Samples were solubilized in ionized water to a concentration of 1 mg/ml.

### Rheometer Characterization

The rheological properties of samples were studied using a Modular Compact Rheometer MCR 92 (Anton Paar GmbH, Graz, Austria), with measure system DG26.7 (double gap geometry) at the temperature of 20° C.

Viscosity measurements were performed in rotation mode, they were investigated in the range of 1-1000 s⁻¹, with a logarithmic ramp, and ten points per decade were acquired.

Samples were solubilized in deionized water to a concentration of 10 mg/ml.

### HP-SEC-TDA

The detector was equipped with Refractive Index, Right and Low Angle Light Scattering and Viscometers detectors. It was used with the following conditions:
Columns: 2 columns TSKGelGPWXL 13 μm. 7.8 mm ID×30 cm L. Tosoh Bioscience.
Mobile phase: 0.3 M AcONa+NaN₃ 0.05%; pH~8.1
Injection volume: 100 μl;
Temperature: 40° C.;
Flow rate: 0.6 ml/min.

The system was calibrated with Pullulan standard, with molecular weight, polydispersity index and intrinsic viscosity certified (PolyCAL-PullulanSTD-Malvern Panalytical).

Samples were solubilized in 0.3 M AcONa+NaN₃ 0.05% (pH-8.1) to a concentration of 1 mg/mL.

### NMR

HSQC-DEPT experiments were performed at Bruker Avance 500 Neo instrument, equipped with 5 mm cryoprobe.

### Acquisition Parameters:

Pulse sequence: hsqcedetgpsisp2.2;
Number of scans (NS): 24;
Pulse delay (D1): 2 s;
Temperature: 313 K;
1J(C,H): 133 Hz.
Sweep width (SW): 7.9 ppm (F2), 160 ppm (F1).
Irradiation frequency: (O1): 4.7 ppm (F2), 80 ppm (F1);
Time domain (TD): 2048 (F2), 320 (F1).

### Processing Parameters:

Spectrum size (SI): 1024 (F2), 1024 (F1)
Window multiplication: shifted QSINE in both dimensions.

¹H spectra were acquired in the following conditions:
Pulse program: zgcppr.ricb
Irradiation frequency (01): 2353.60 Hz
Spectral width (SW): 18 ppm
Time domain (TD): 32768
Pulse delay (D1):12 s
Number of scans (NS): 16
Temperature: 313 K

¹³C spectra were acquired in the following conditions:
Pulse program: zggppgse.t1.ez
Irradiation frequency (O1): 12576.11 Hz
Spectral width (SW): 294 ppm
Time domain (TD): 32768
Pulse delay (D1):1 s
Number of scans (NS): 10000
Temperature: 313 K

Samples were solubilized in Deuterium oxide (D₂O) to a concentration of 8-10 mg/ml TITRATOR

Conductimetric titration was performed using 888 Titrando system. It was utilized under the following conditions:
Titrating agent: NaOH 0.1N
Volume increment: 0.150 mL
Stop volume: 10 mL
Stirring rate: 4

About 150 mg of each sample were solubilized in the right amount of deionized water to obtain a homogenous solution. Then, the solutions were transformed into its acid form using a ion exchange resin (Amberlite IR-120(H⁺)) and then titrated by addition of sodium hydroxide solution 0.1N.

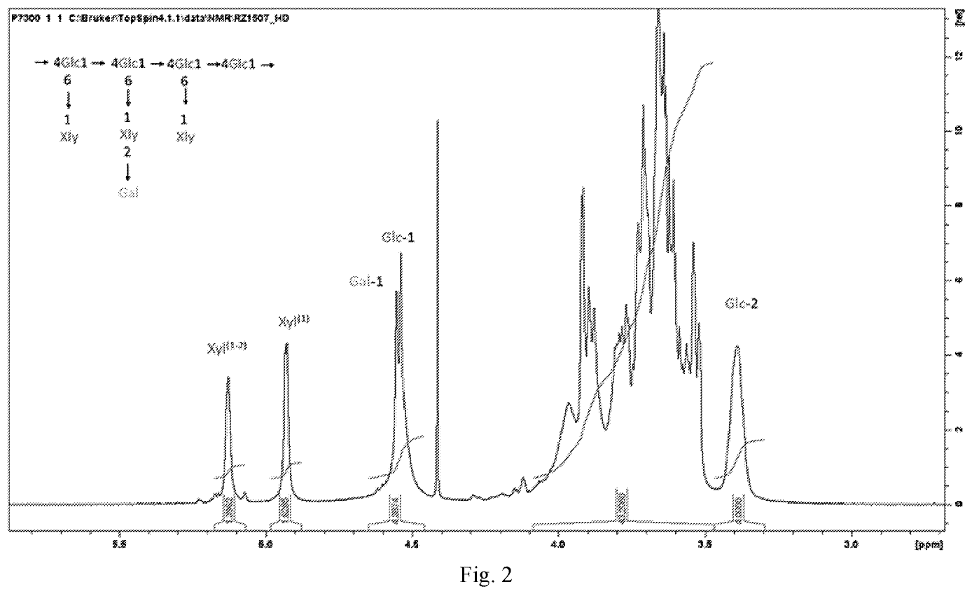

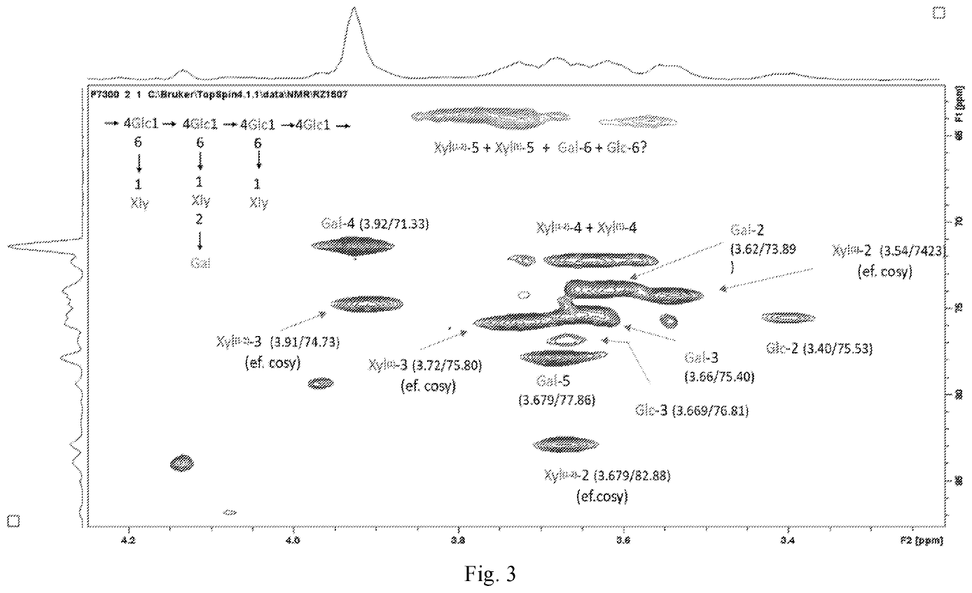

In FIG. 1, the structure of the octasaccharide repeating unit of the native TSP, while FIG. 2 shows ¹H NMR spectrum of native TSP and FIG. 3 shows HSQC-DEPT spectrum of native TSP

### Example 1

Tamarind seed polysaccharide (500 mg; 0.41 mmol; 1 eq.) was suspended in dry dimethylformamide (DMF, 50 ml), the mixture was stirred overnight at room temperature. 1.38 g (8.7 mmoli; 1 eq.) of sulphur trioxide-pyridine complex (SO₃-Py) was then added to the. To the resulting finely dispersed suspension was stirred vigorously for 24 h at room temperature under atmospheric pressure. After dilution with water (10 ml) and cooling to room temperature, the homogeneous mixture was obtained, the solution pH was adjusted to 6-7 with NaOH solution (1N), and then precipitated with alcohol (EtOH/H₂0 70% v/v) and the sulphated TSP recovered by centrifugation. The solid was dissolved in water (40 ml) and after careful neutralisation with sodium hydroxide solution, precipitated again with ethanol 70% v/v) to remove DMF, Pyridine and salt excess of sulphating agent and potential degradation production of the latter (Na₂SO₄) before being freeze-dried. Were obtained 595 mg of sulphated TSP (shortly referred to as “P7351”), meaning a yield of about 95%. Barium chloride Test: Negative

The achievement of sulphated sample (TSPS-P7351) was confirmed by ¹³C-NMR spectra and conductimetric titration. The results showed that TSP was sulphated to give a sulphate polysaccharide P7351 with a degree of substitution DS₍ₘₒₗₑ₎=3.16

5.31 mg of P7351 were then solubilized in 0.6 mL of D₂O for NMR study.

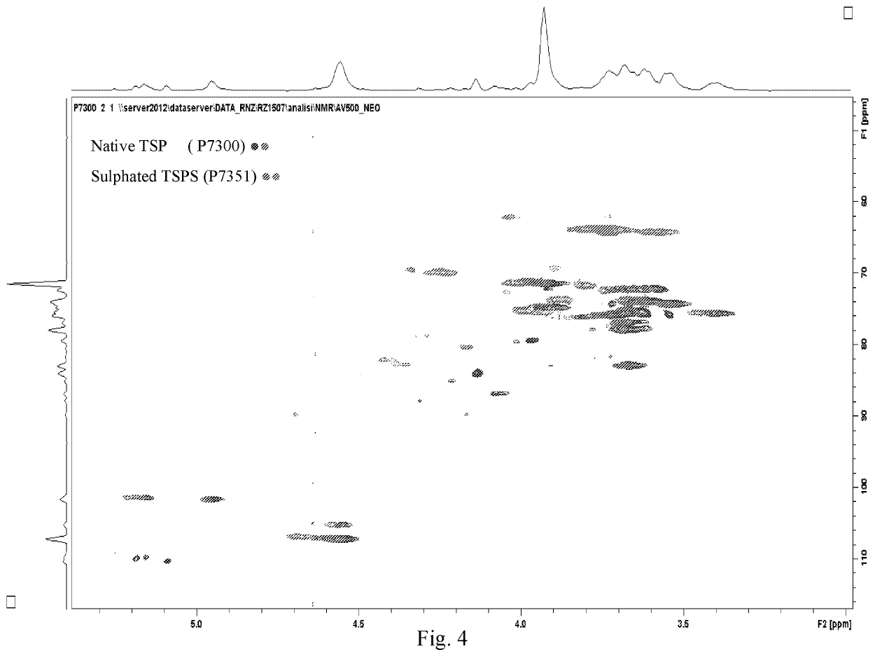

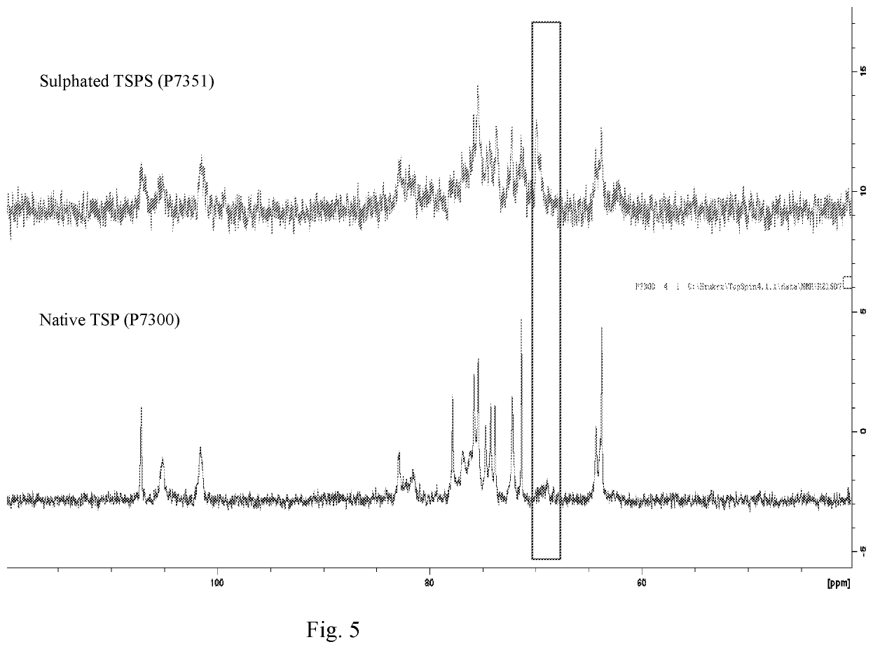

In FIG. 4 and FIG. 5, the HSQC-DEPT and ¹³C spectra of P7300 and P7351 are compared. The signals of the sample P7351 are shifted compared to those of P7300, due the presence of the sulphate group. The carbons directly bound to sulphonic groups might shift to lower field position, while others indirectly bound to sulphonic groups would shift to higher field position. The decreased intensity of the peak at δ 69.97 ppm indicated that the hydroxy group on C-6 of galactose and unsubstituted backbone glucose residues was partially substituted by a sulphonic group.

The substitution degree (DS) was determined via conductimetric titration, as follows. 140 mg of P7351 were converted into its free acid by using an ion exchange resin (Amberlite IR-120(H⁺)) and then titrated by addition of sodium hydroxide solution 0.1N. The number of sulphate groups on the repeating polysaccharide unit calculated is 3.16, which corresponds to 15.1% of the total hydroxyl groups (21) that can be substituted within the repeating unit of the TSP shown in FIG. 1.

The Zeta Potential of P7300 and P7351 was also determined via dynamic light scattering, as follows.

5 mg of P7300 were solubilized in 10 mL of deionized water and 3 mg of P7351 were dissolved in 3 mL of deionized water. TSP is a neutral polysaccharide and, in fact, its Zp value is −0.356 mV. Instead, P7351 has a Zp value of −38.2 mV, due to the negative charges of the sulphate groups.

Molecular weight of P7300 and P7351 was evaluated via HP-SEC-TDA, as follows. 5 mg of both samples were solubilized in 5 mL of AcONa 0.3M+NaN₃ 0.05% (pH~8.1). The molecular weight of P7300 is 613 kDa, whereas the molecular weight of P7351 is 965 kDa. The increase of the molecular weight of the derivative P7351 is ascribed to the presence of the sulphate groups.

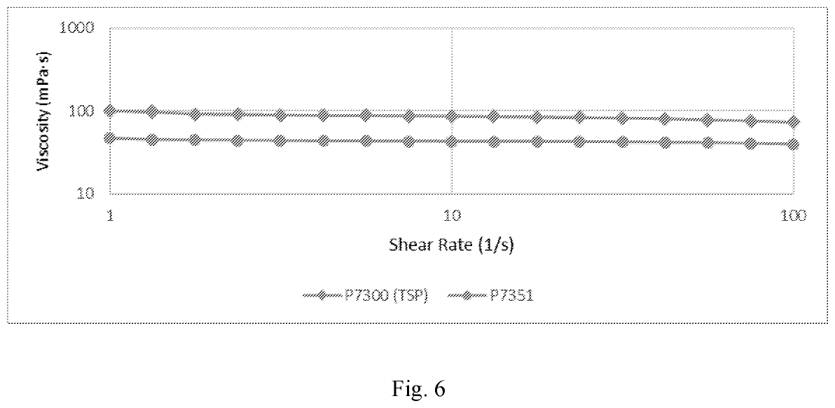

To determinate the viscosity of the solutions, 100 mg of P7300 and P7351 were solubilized both in 10 mL of deionized water. The variations of viscosity as a function of the shear rate were acquired for P7300 and P7351 by using double gap geometry; the viscosity curves, in the shear rate (γ) range 1 s⁻¹ to 100 s⁻¹ are reported for both products at the temperature of 20° C., as shown in FIG. 6.

Native TPS P7300 shows a higher viscosity than sulphated P7351: at the shear rate of 10 l/s, P7300 has a viscosity of 86.4 mPa s whereas P7351 has a viscosity of 42.7 mPa·s. This difference may be ascribed to the presence of charged groups, i.e. sulphate groups, which affect the structural density of the TSP.

### Example 2

Tamarind seed polysaccharide (500 mg; 0.41 mmol; 1 eq.) was suspended in dry dimethylformamide (DMF, 50 ml), the mixture was stirred overnight at room temperature. 1.38 g (8.7 mmoli; 1 eq.) of sulphur trioxide-pyridine complex (SO₃-Py) was then added to the. To the resulting finely dispersed suspension was stirred vigorously for 24 h at 50° C. atmospheric pressure. After dilution with water (10 ml) and cooling to room temperature, the homogeneous mixture was obtained, the solution pH was adjusted to 6-7 with NaOH solution (1N), and then precipitated with alcohol (EtOH/H₂O 70% v/v) and the sulphated TSP recovered by centrifugation. The solid was dissolved in water (40 ml) and after careful neutralisation with sodium hydroxide solution, precipitated again with ethanol 70% v/v) to remove DMF, pyridine and salt excess of sulphating agent and potential degradation production of the latter (Na₂SO₄) before being freeze-dried. Were obtained 557 mg of sulphated TSP (shortly referred to as “P7352”), meaning a yield of about 94%. Barium chloride Test: Negative

The linked ester sulphate group content in the P7352 sample was confirmed by ¹³C-NMR spectra and conductimetric titration. Said analyses showed that TSP was sulphated to give a sulphate polysaccharide P7352 with a degree of substitution DS₍ₘₒₗₑ₎=2.29.

6.5 mg of P7352 were then solubilized in 0.6 mL of D₂O for NMR study.

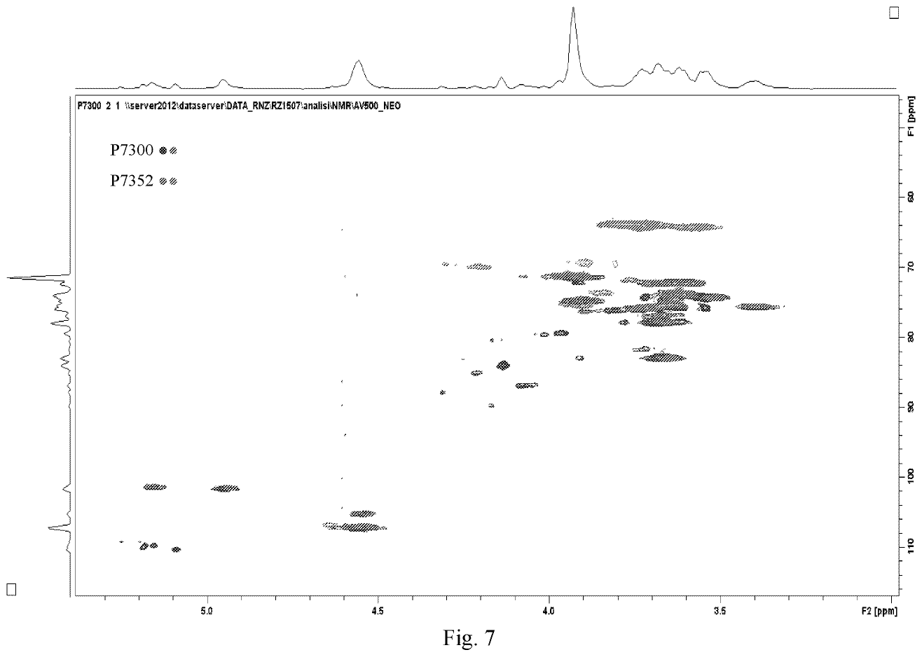

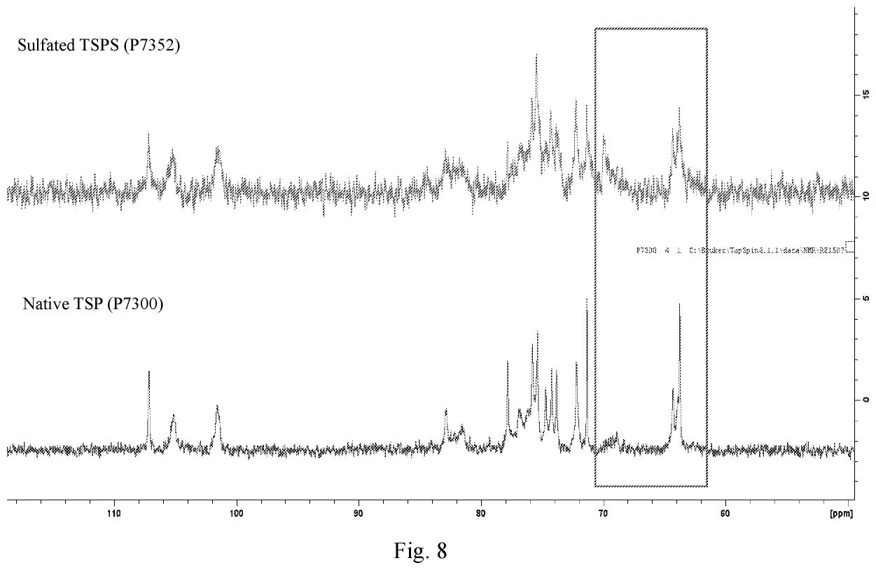

In FIG. 7 and FIG. 8, the HSQC-DEPT and ¹³C spectra of P7300 and P7351 are compared. ¹³C-NMR spectrum of the sulphated sample P7352 shows the presence of a new signal at δ=69.93, indicating the substitution of —CH₂OH groups after sulphation reaction.

The substitution degree (DS) was determined via conductimetric titration, as follows. 150 mg of P7352 were converted into its free acid by using an ion exchange resin (Amberlite IR-120(H⁺)) and then titrated by addition of sodium hydroxide solution 0.1N. The number of sulphate groups on the repeating polysaccharide unit calculated is 2.3, which corresponds to 11% of the total hydroxyl groups (21) that can be substituted within the repeating unit of the TSP shown in FIG. 1.

The Zeta Potential of P7352 was also determined via dynamic light scattering, as follows. 3 mg of P7352 were dissolved in 3 mL of deionized water. P7352 has a Zp value of −37.8 mV, due to the negative charges of the sulphate groups.

Molecular weight of P7352 was evaluated via HP-SEC-TDA, as follows.

5 mg of the sample were solubilized in 5 mL of AcONa 0.3M+NaN₃ 0.05% (pH~8.1). The molecular weight of P7352 is 804 kDa. The increase of the molecular weight of the derivative P7352 is ascribed to the presence of the sulphate groups.

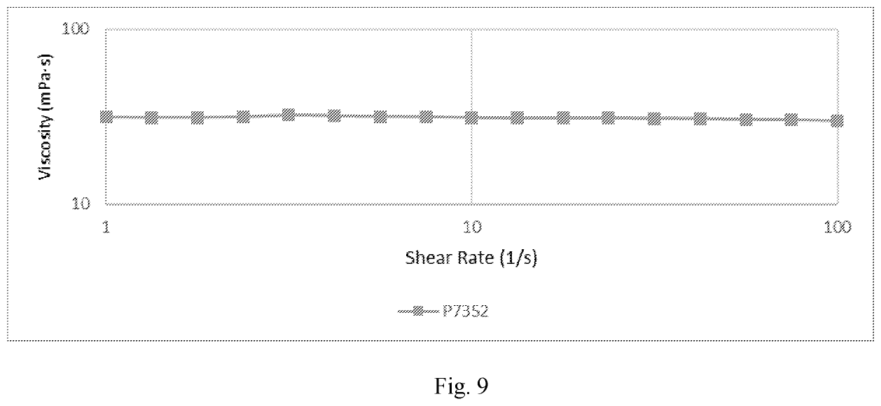

To determinate the viscosity of the solutions, 100 mg of P7352 were solubilized in 10 mL of deionized water. The variations of viscosity as a function of the shear rate were acquired for P7352 by using double gap geometry; the viscosity curves, in the shear rate (γ) range 1 s⁻¹ to 100 s⁻¹ are reported at the temperature of 20° C., as shown in FIG. 9.

P7352 has a viscosity of 31.2 mPa s at the shear rate of 10 l/s.

### Example 3

Tamarind seed polysaccharide (500 mg; 0.41 mmol; 1 eq.) was suspended in dry dimethylformamide (DMF, 50 ml), the mixture was stirred overnight at room temperature. 1.33 g (8.7 mmoli; 1 eq.) of sulphur trioxide-N,N′dimethylformamide complex (SO₃-DMF) was then added to the. To the resulting finely dispersed suspension was stirred vigorously for 24 h at room temperature and atmospheric pressure. After dilution with water (10 ml) and cooling to room temperature, the homogeneous mixture was obtained, the solution pH was adjusted to 6-7 with NaOH solution (1N), and then precipitated with alcohol (EtOH/H₂O 70% v/v) and the sulphated TSP recovered by centrifugation. The solid was dissolved in water (40 ml) and after careful neutralisation with sodium hydroxide solution, precipitated again with ethanol 70% v/v) to remove DMF, pyridine and salt excess of sulphating agent and potential degradation production of the latter (Na₂SO₄) before being freeze-dried. Were obtained 515 mg of sulphated TSP (shortly referred to as “P7353”), meaning a yield of about 94%. Barium chloride Test: Negative

The linked ester sulphate group content in the P7353 sample was confirmed by ¹³C-NMR spectra and conductimetric titration. The results showed that TSP was sulphated to give a sulphate polysaccharide P7353 with a degree of substitution DS₍ₘₒₗₑ₎=1.27.

5.34 mg of P7353 were solubilized in 0.6 mL of D₂O for NMR study.

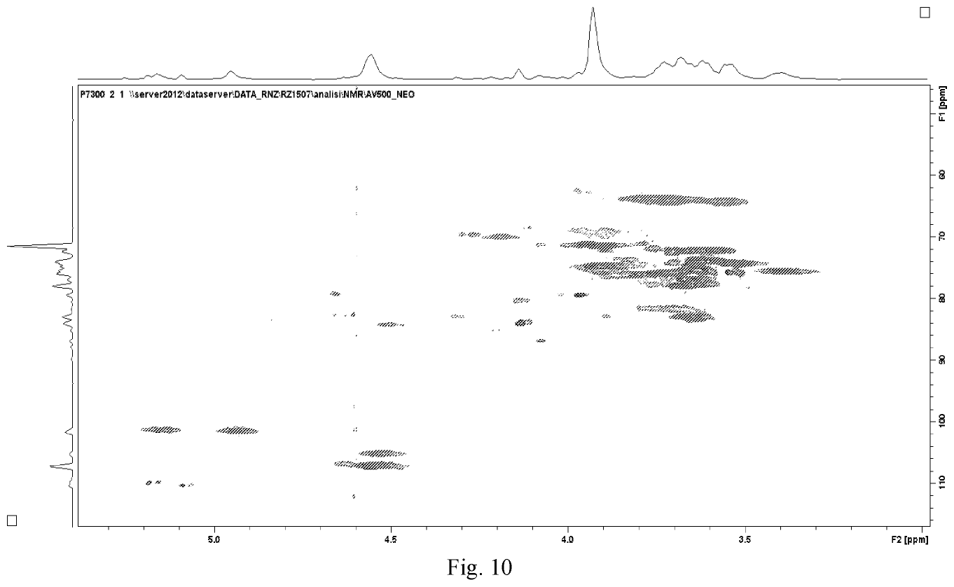

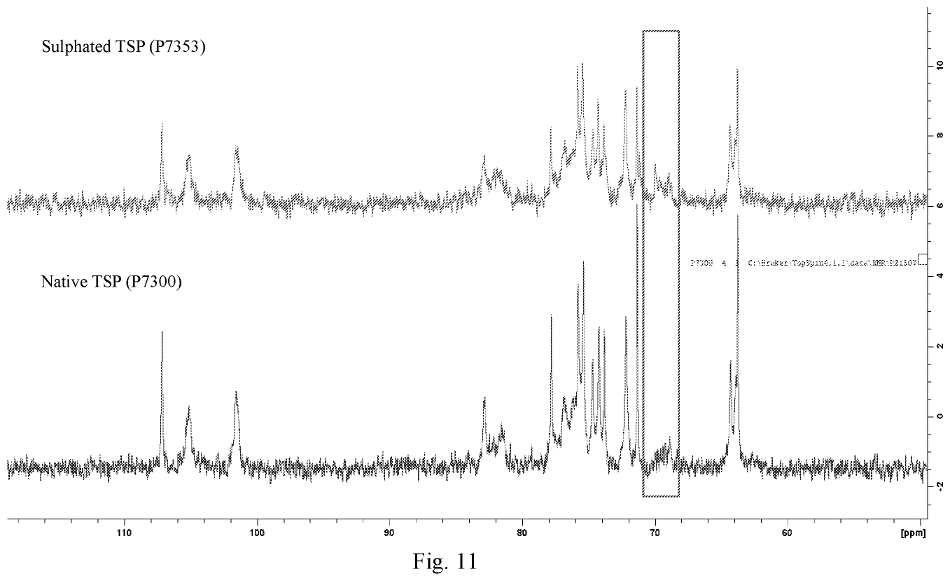

In FIG. 10 and FIG. 11, the HSQC-DEPT spectra of P7300 and P7352 are compared.

The signals of the sample P7353 are shifted compared to those of P7300, due the presence of the sulphate group.

The substitution degree (DS) was determined via conductimetric titration, as follows. 150 mg of P7353 were converted into its free acid by using an ion exchange resin (Amberlite IR-120(H+)) and then titrated by addition of sodium hydroxide solution 0.1N. The number of sulphate groups on the repeating polysaccharide unit calculated is 1.2, which corresponds to 5.7% of the total hydroxyl groups (21) that can be substituted within the repeating unit of the TSP shown in FIG. 1.

The Zeta Potential of P7353 was also determined via dynamic light scattering, as follows. 3 mg of P7353 were dissolved in 3 mL of deionized water. P7353 has a Zp value of −24.6 mV, due to the negative charges of the sulphate groups.

Molecular weight of P7353 was evaluated via HP-SEC-TDA, as follows. 5 mg of the sample were solubilized in 5 mL of AcONa 0.3M+NaN₃ 0.05% (pH~8.1). The molecular weight of P7353 is 804 kDa. The increase of the molecular weight of the derivative P7353 is ascribed to the presence of the sulphate groups.

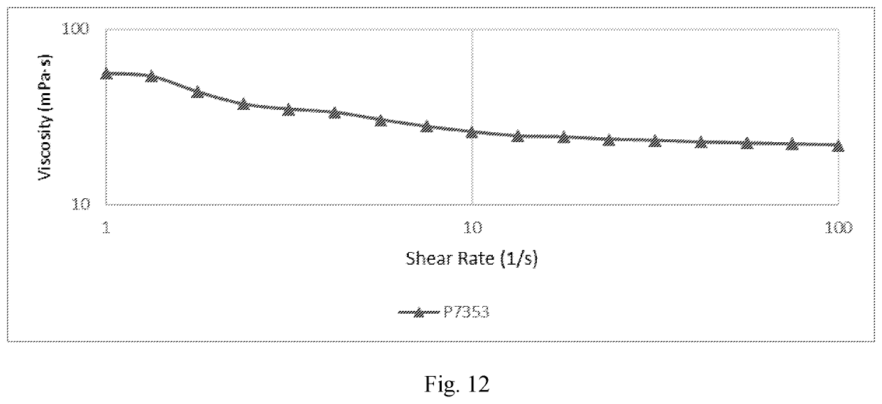

To determinate the viscosity of the solutions, 100 mg of P7353 were solubilized in 10 mL of deionized water. The variations of viscosity as a function of the shear rate were acquired for P7353 by using double gap geometry; the viscosity curves, in the shear rate (γ) range 1 s⁻¹ to 100 s⁻¹ are reported at the temperature of 20° C., as shown in FIG. 12.

P7353 has a viscosity of 26 mPa s at the shear rate of 10 l/s.

1. A sulphated tamarind seed polysaccharide (TSP) having a weight average molecular weight of 600-1′500 kDa, and a degree of sulphation (DS) of 1 to 10 per octasaccharide repeating unit, said DS being determined via conductimetric titration and calculated according to the following formula:
$$D {S}_{( m o l e )} = \frac{mmole NaOH}{\frac{mg TSP - ( mmole NaOH \times 80 g / mole )}{1207 g / mole}}$$
where NaOH is the titrating agent.

2. The sulphated tamarind seed polysaccharide of claim 1, having a weight average molecular weight of 700-1′ 100 kDa, and having a degree of sulphation (DS) of 1 to 5 per octasaccharide repeating unit.

3. The sulphated tamarind seed polysaccharide of claim 1, having a viscosity of 20 to 60 mPa*s, at a shear rate of 10 l/s and at a temperature of 20° C.

4. The sulphated tamarind seed polysaccharide of claim 1, having a Zeta potential of −20.00 to −50.00 mV, as measured by Dynamic Light Scattering (DLS).

5. The sulphated tamarind seed polysaccharide of claim 1, being salified with a heavy metal or with a pharmacologically active substance.

6. The sulphated tamarind seed polysaccharide of claim 5, wherein the heavy metal is a metal element selected from the 4th, 5th and 6th groups of the periodic table of elements.

7. The sulphated tamarind seed polysaccharide of claim 6, wherein said heavy metal is silver, cobalt, iron, copper, zinc, arsenic, strontium, zirconium, antimony, gold, caesium, tungsten, selenium, platinum, ruthenium, bismuth, tin, titanium, or mercury.

8. The sulphated tamarind seed polysaccharide of claim 5, wherein the pharmacologically active substance is selected from the group consisting of an antibiotic, an anti-infective, an antimicrobial, an antiviral, a cytostatic, an antitumoral, an anti-inflammatory, a wound healing agent, an anaesthetic, a cholinergic antagonist, an adrenergic agonist, an antithrombotic, an anticoagulant, a haemostatic, a fibrinolytic, a thrombolytic agent, a protein, a protein fragment, a peptide, and a polynucleotide.

9. A pharmaceutical composition comprising the sulphated tamarind seed polysaccharide of claim 1 and pharmaceutically acceptable excipients.

10.-11. (canceled)

12. A process for preparing the sulphated tamarind seed polysaccharide of claim 1, the process comprising the steps of:
i) providing a suspension of TSP in a tertiary amine solvent,
ii) adding a complex of sulphur trioxide with an organic radical selected from the group consisting of pyridine, DMF, trimethylamine, dioxane, N,N-dimethylaniline, B′,B-dichlorodiethyl ether, and mixture thereof, under stirring at a temperature of 15-60° C., thus obtaining a dispersion of sulphated TSP,
iii) adding water to said dispersion, thus obtaining a homogenous mixture, and adjusting the pH to 6-7,
iv) adding a alcoholic solvent to said mixture, for promoting the precipitation of the sulphated TSP, and
v) separating and purifying the sulphated TSP.

13. The process of claim 12, wherein, in step i), said tertiary amine solvent is selected from DMF (dimethylformamide), pyridine, trimethylamine, picolines, N,N-dimethylaniline, quinolines, and mixtures thereof.

14. The process of claim 12, wherein, in step ii), wherein said complex and TSP are in a molar ratio of 1:1 to 1:10.

15. The process of claim 12, wherein, in step iv), said alcoholic solvent is methyl alcohol, ethyl alcohol, propyl alcohol, or a mixture thereof, optionally diluted with water.
